(19) United States
(12) Patent Application Publication
(10) Pub. No.: US 2024/0358655 A1
(43) Pub. Date: Oct. 31, 2024

(54) Transdermal Systems having low dose estrogen and methods of making and use

(71) Applicant: Viatris Inc., Canonsburg, PA (US)

(72) Inventors: David P. Kanios, Palmetto Bay, FL (US); Gaurav Tolia, Morgantown, WV (US)

(73) Assignee: Viatris Inc., Canonsburg, PA (US)

(21) Appl. No.: 18/553,368

(22) PCT Filed: Mar. 30, 2022
(86) PCT No.: PCT/US2022/022580

§ 371 Date: Sep. 29, 2023

Related U.S. Application Data
(60) Provisional application No. 63/167,967, filed on Mar. 30, 2021.

(51) Int. Cl. A61K 9/70; A61K 31/567

(52) U.S. Cl. CPC A61K 9/7053; A61K 31/567

(57) ABSTRACT

A transdermal system for providing estrogen to a patient in need thereof is provided. The transdermal system comprises a backing, and a matrix contacting the backing, the matrix configured to release about 4 meg per day to about 36 meg per day of an estrogen to the patient. This application also provides methods of providing estrogen to a patient in need thereof. The methods described in this application comprise applying to the skin of the patient a transdermal system comprising a backing, and a matrix contacting the backing, the matrix configured to release about 4 meg per day to about 36 meg per day of an estrogen to the patient.

### TECHNICAL FIELD

This application relates to transdermal systems for drug delivery. More particularly, it concerns transdermal systems and methods for transdermally administering a low dose of an estrogen, particularly ethinyl estradiol, either alone or in combination with a progestin such as norelgestromin.

### BACKGROUND

One of the leading pharmaceutical methods currently used for the prevention of pregnancy involves the administration of a combination of an estrogen and a progestin to a pre-menopausal woman in the form of a transdermal system, such as for example, a transdermal patch or film. The use of the desired contraceptive agents has been well developed using a solid oral dosage form, such as a tablet or capsule.

However, the parameters around the use of a transdermal system to deliver contraceptive agents are not well understood, despite the advantages of a transdermal system over a solid oral dosage form in terms of enhanced patient compliance. Currently, only ORTHO-EVRA® and TWIRLA® transdermal systems have been approved as new drug applications by the U.S. F.D.A. for the prevention of pregnancy.

The first contraceptive patch, ORTHO-EVRA®, was approved in 2001 and is labeled with a delivery rate of 35 micrograms (mcg) ethinyl estradiol (EE) and 150 mcg norelgestromin per day; XULANE® is a generic equivalent to ORTHO-EVRA®. The TWIRLA® transdermal system is labeled with a delivery rate of 30 mcg ethinyl estradiol/120 mcg levonorgestrel per day.

The labels for both TWIRLA® and ORTHO-EVRA®, and now XULANE®, are required by the U.S. F.D.A to contain a black-box warning. In 2011, the ORTHO-EVRA® labelling was amended to warn consumers of the potentially higher risk of venous thromboembolism events (VTEs) compared to oral contraceptives. The label further stated that increased estrogen exposure may increase the risk of serious adverse events, including VTE. TWIRLA® was approved with the current warning in the label in 2020. The current warning cautions that the use of the TWIRLA® or XULANE® transdermal system is contraindicated for women with a BMI≥30 because of a potentially higher risk of VTE compared to women with a lower BMI. Therefore, women with a BMI≥30 have not been able to use a transdermal system for birth control since 2020. The need to maintain sufficient plasma levels of oral contraceptives to prevent pregnancy directly opposes the reduction of the hormones in the commercial transdermal products that might result in a lower risk of VTEs for women with a BMI≥30.

A transdermal system has a much different mechanism of drug delivery than an immediate-release oral solid dosage form. In a transdermal system, the active agents pass directly through the epidermal layer and are absorbed into the bloodstream. Additionally, the release of the active agents from the transdermal system is generally engineered to be fairly constant over time, such that the plasma availability of the active agents is steady and constant.

However, the pharmacokinetic profile of an oral dosage form such as a tablet or capsule typically reflects two distinct events associated with degradation of the medicament in the gastrointestinal tract. The first event typically involves the appearance of a “spike” in blood levels of the active agents, reflecting the initial fast dissolution and/or disintegration of the active agents from the tablet. The spike is followed by a flatter line that eventually decreases to zero, a result of the relatively slower absorption of the active agents as the tablet crodes. As a result, the plasma levels of the active agents vary greatly over the course of the day.

Additionally, while the dosing regimen for an immediate release contraceptive tablet is one tablet or capsule per day, a transdermal system for contraception can release the ethinyl estradiol/progestin combination for a full seven days per administration of each film or patch. Therefore, while the transdermal contraceptive system delivers a relatively flat plasma level of ethinyl estradiol and norelgestromin over seven days, a contraceptive tablet or capsule will have provided seven distinct pharmacokinetic peak levels and valley levels of the contraceptive agents.

Because of the difference in pharmacokinetic profiles provided by the different dosage forms, the amount of ethinyl estradiol used in a tablet or capsule cannot predict the amount of efficacious ethinyl estradiol that can be delivered daily by a transdermal system.

It would therefore be beneficial to provide a transdermal system (e.g., transdermal film or patch) with reduced amounts of the hormones used to prevent pregnancies. It would also be desirable to provide a transdermal delivery system that can deliver the contraceptive hormones steadily at a consistent rate that does not vary greatly over a long period of time.

### SUMMARY

This application provides transdermal systems (e.g., transdermal film or patch) and methods for preventing ovulation by the administration of an effective, low dose of an estrogen, particularly ethinyl estradiol, either alone or in combination with a progestin such as norelgestromin.

In some embodiments, there is a transdermal system for releasing a contraceptive to a patient in need thereof, the transdermal system comprising a backing, and a matrix contacting the backing, the matrix configured to release about 4 meg per day to about 28 mcg per day of an estrogen to the patient. In some embodiments, the estrogen (e.g., ethinyl estradiol) can be in the transdermal systems (e.g., transdermal film or patch) as the only active pharmaceutical ingredient. In some embodiments, the estrogen (e.g., ethinyl estradiol) can be in the transdermal systems (e.g., transdermal film or patch) with a progestin (e.g., norelgestromin).

In various embodiments, this application provides a method of providing contraception to a patient in need thereof. The methods described in this application comprise applying to the skin of the patient a transdermal system comprising a backing, and a matrix contacting the backing, the matrix configured to release about 4 meg per day to about 28 meg per day of an estrogen to the patient.

In some embodiments, there is a method of making a transdermal system, the method comprising mixing about 0.1 mg to about 0.396 mg of ethinyl estradiol, or the therapeutic equivalent of an alternative estrogen, with an adhesive to create the transdermal system, wherein the transdermal system is configured to release about 4 meg per day to about 28 mcg per day of an estrogen to a patient.

In some embodiments, there is a transdermal system for releasing a contraceptive to a patient in need thereof, the transdermal system comprising a backing, and a matrix contacting the backing, the matrix configured to release about 4 meg per day to about 28 mcg per day of an estrogen to the patient.

In some embodiments, there is a method of providing contraception to a patient in need thereof, the method comprising applying to skin of the patient a transdermal system comprising a backing, and a matrix contacting the backing, the matrix configured to release about 4 mcg per day to about 28 meg per day of an estrogen to the patient.

In some embodiments, there is a method of making a transdermal system, the method comprising mixing about 0.21 mg to about 0.48 mg of estrogen with an adhesive and applying it to the transdermal system, wherein the transdermal system is configured to release about 4 mcg per day to about 28 meg per day of an estrogen to a patient.

In some embodiments, there is a transdermal system for providing an estrogen to a patient in need thereof, the transdermal system comprising a backing, and a matrix contacting the backing, the matrix configured to release about 4 meg per day to about 28 meg per day of an estrogen to the patient, wherein the estrogen is the only active pharmaceutical ingredient in the transdermal system.

In some embodiments, there is a method of providing an estrogen to a patient in need thereof, the method comprising applying to skin of the patient a transdermal system comprising a backing, and a matrix contacting the backing, the matrix configured to release about 4 meg per day to about 28 meg per day of an estrogen to the patient, wherein the estrogen is the only active pharmaceutical ingredient in the transdermal system.

In some embodiments, there is a method of making a transdermal system, the method comprising mixing about 0.21 mg to about 0.48 mg of an estrogen with an adhesive and applying it to the transdermal system, wherein the transdermal system is configured to release about 4 mcg per day to about 28 meg per day of an estrogen to a patient, and the estrogen is the only active pharmaceutical ingredient in the transdermal system.

In some embodiments, there is a method for inhibiting ovulation in a patient in need thereof, the method comprising providing a transdermal system for releasing an estrogen to a patient in need thereof, the transdermal system comprising a backing, and a matrix contacting the backing, the matrix configured to release about 4 meg per day to about 28 meg per day of the estrogen to the patient and applying the transdermal system to skin of the patient.

In some embodiments, there is a transdermal system for releasing a contraceptive to a patient in need thereof, the transdermal system comprising a backing, and a matrix contacting the backing, the matrix configured to release about 4 meg per day to about 28 mcg per day of an estrogen to the patient, wherein pregnancy of a human female patient is prevented and the human female patient has a body mass index (BMI) of less than 30 kg/m².

In some embodiments, there is a transdermal system for releasing a contraceptive to a patient in need thereof, the transdermal system comprising a backing, and a matrix contacting the backing, the matrix configured to release about 4 meg per day to about 28 meg per day of an estrogen to the patient, wherein pregnancy of a human female patient is prevented and the human female patient has a body mass index (BMI) of greater than or equal to 30 kg/m².

In some embodiments, there is a transdermal system for releasing a contraceptive to a patient in need thereof, the transdermal system comprising a backing, and a matrix contacting the backing, the matrix configured to release about 4 meg per day to about 28 meg per day of an estrogen to the patient, wherein the patient has a lower risk of venous thromboembolism events as compared to a patient receiving more than 28 meg per day of estrogen.

In some embodiments, there is a method of treating a condition responsive to an estrogen, the method comprising applying to skin of a patient a transdermal system comprising a backing, and a matrix contacting the backing, the matrix configured to release about 4 mcg per day to about 36 mg per day of an estrogen to the patient.

In one aspect of the application, a transdermal patch for administering norelgestromin and a low dose estrogen to a woman is provided, the patch comprising a backing and a matrix underlying the backing, the matrix comprising a mixture of norelgestromin, a low dose estrogen, and a pressure sensitive adhesive, and being adapted to be in diffusional communication with the skin of the woman and to co-administer an ovulation-inhibiting amount of said norelgestromin and low dose estrogen to the woman through the skin.

Another aspect of this application is a transdermal patch for preventing ovulation in a woman comprising a backing and a matrix underlying the backing, the matrix comprising a mixture of norelgestromin, a low dose of an estrogen, and a pressure sensitive adhesive and being adapted to be in diffusional communication with the skin of the woman and to administer an ovulation-inhibiting amount of norelgestromin and the low dose estrogen to said skin.

In some embodiments, it is contemplated that the transdermal system provided can adequately serve to prevent pregnancy while simultaneously allowing women with a BMI≥30 to utilize the current transdermal system.

While multiple embodiments are disclosed, still other embodiments of the present disclosure will become apparent to those skilled in the art from the following detailed description. As will be apparent, the disclosure is capable of modifications in various obvious aspects, all without departing from the spirit and scope of the present disclosure. Accordingly, the detailed description is to be regarded as illustrative in nature and not restrictive.

### BRIEF DESCRIPTION OF THE FIGURES

In part, other aspects, features, benefits and advantages of the embodiments will be apparent with regard to the following description, appended claims, and accompanying drawings.

FIG. 1 illustrates graphs of mean norelgestromin plasma concentrations in transdermal delivery systems delivering per day: (A) about 19 mcg ethinyl estradiol and about 205 mcg norelgestromin; (B) about 28 meg ethinyl estradiol and about 205 meg norelgestromin; and (C) about 35 mcg ethinyl estradiol and about 150 mcg norelgestromin (the commercial XULANE® transdermal system product).

FIG. 2 illustrates graphs of mean ethinyl estradiol plasma concentrations in transdermal delivery systems delivering per day: (A) about 19 mcg ethinyl estradiol and about 150 mcg norelgestromin; (B) about 28 meg ethinyl estradiol and about 205 meg norelgestromin; and (C) about 35 mcg ethinyl estradiol and about 205 mcg norelgestromin (the commercial XULANE® transdermal system film).

FIG. 3 illustrates pharmacokinetic profiles of a transdermal delivery system (A), delivering per day about 19 mcg ethinyl estradiol and 205 mcg norelgestromin, and an immediate-release tablet containing 25 mcg cthinyl estradiol and 180 mcg norgestimate.

FIG. 4 illustrates graphs of a mean serum ethinyl estradiol concentrations in healthy female volunteers following two consecutive cycles of TWIRLAR wear on the buttock where the vertical arrow indicates time of TWIRLA® removal.

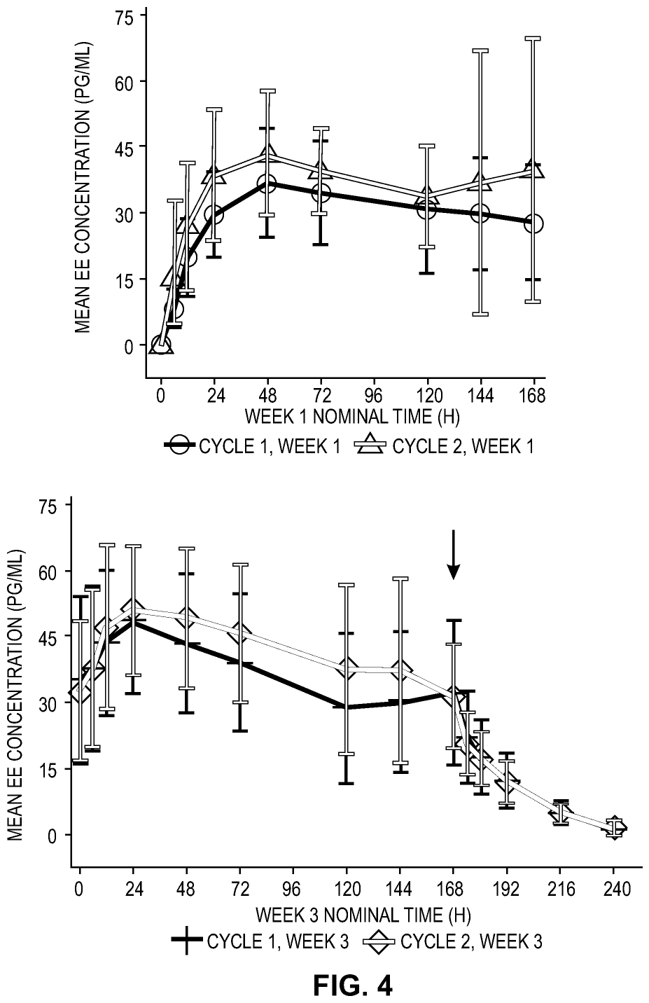

FIG. 5 illustrates graphs of mean serum levonorgestrel concentrations in healthy female volunteers following two consecutive cycles of TWIRLA® wear on the buttock where the vertical arrow indicates time of TWIRLA® removal.

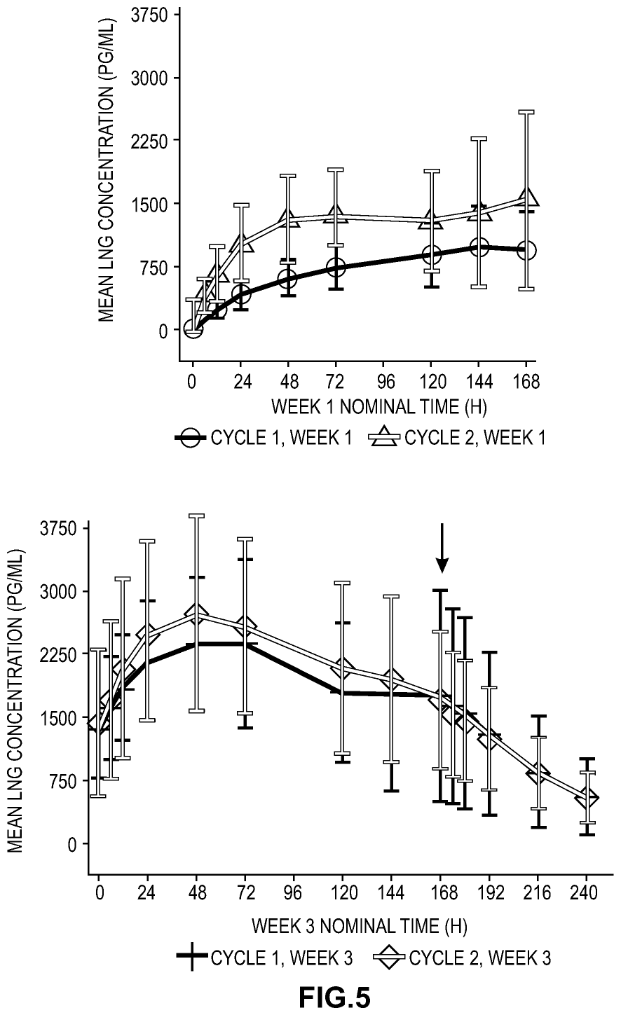

FIG. 6 is a schematic diagram of a transdermal delivery system of norelgestromin (NGMN) and ethinyl estradiol (EE).

FIG. 7 is a graph illustrating mean EE serum concentrations in pg/mL in healthy female volunteers following application of a commercially available ORTHO EVRA® transdermal system applied to the buttocks for three consecutive cycles. The dotted horizontal lines indicate the reference range. The dotted vertical arrow indicates time of patch removal.

FIG. 8 is a graph illustrating mean NGMN serum concentrations in ng/ml in healthy female volunteers following application of a commercially available ORTHO EVRA® transdermal system applied to the buttocks for three consecutive cycles. The dotted horizontal lines indicate the reference range. The dotted vertical arrow indicates time of patch removal.

FIG. 9 illustrates linear and semi log scale graphs of mean norelgestromin plasma concentrations produced by treatment groups A, B, and C of Table 1.

FIG. 10 illustrates linear and semi log scale graphs of mean ethinyl estradiol plasma concentrations produced by treatment groups A, B, and C of Table 1.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

This application provides a pharmaceutical composition that can be used in a transdermal delivery system, which includes a transdermal system (e.g., film or patch) intended as a method for preventing ovulation in a woman. In recent years, efforts have been made to reduce the amount of ethinyl estradiol present in the oral solid dosage forms in order to reduce side effects. However, these efforts have been largely targeted at the standard immediate-release dosage forms. In some embodiments, the transdermal delivery system used for this method is a transdermal patch, comprising a backing and a matrix contacting the backing, where the matrix comprises a pressure sensitive adhesive, a low dose of an estrogen, and optionally a progestin such as progestromin and, and is adapted to be in diffusional communication with the skin of the woman and to administer the low dose estrogen and, if present, an ovulation-inhibiting amount of the progestin, to said skin. In various embodiments, the matrix is configured to release about 4 meg per day to about 36 mcg per day of estrogen to a patient. In various embodiments, the matrix is configured to release about 5 mcg per day to about 28 meg per day of estrogen to a patient. In other embodiments, the transdermal system of this application is configured to release estrogen in an amount of (i) 5 to 28 mcg/day; (ii) about 10 to about 27 mcg/day; (iii) about 15 to about 20 mcg/day; or (iv) about 18 to 22 mcg/day. In some embodiments, the matrix further comprises progestin, for example, norelgestromin.

In various embodiments, the backing is in the form of a layer and is impermeable to estrogen. The matrix comprises an adhesive to permit contact with the skin of a patient and allow the estrogen to be released from the matrix through the skin of the patient.

The effective dose of norelgestromin, when used with an estrogen, for inhibiting ovulation normally varies in a range of from about 100, 105, 110, 115, 120, 125, 130, 140, 145, 150, 155, 160, 165, 170, 175, 180, 190, 200, 205, 210, 220, 230, 240, 250, 260, 270, 280, 290, 300, 310, 320, 330, 340 to about 350 meg/day, in some aspects, from about 125 to about 300 mcg/day, and in other aspects, from about 140 to about 200 mcg/day. In some embodiments, the effective dose is from about 170 to about 230 mcg/day. The effective dose of norelgestromin is to be administered in conjunction with other active ingredients including an estrogen.

The effective dose of levonorgestrel for inhibiting ovulation, when used with estrogen, normally varies in a range of about 50, 60, 70, 80, 90, 100, 110, 120, 130, 140, 150, 160, 170, 180, 190, 200, 205, 210, 220, 230, 240, 250, 260, 270, 280, 290 to about 300 mcg/day, in some aspects, from about 100 to about 200 mcg/day, and in other aspects from about 100 to about 150 mcg/day. In some embodiments, the effective dose is from about 190 mcg to about 210 mcg/day. The effective dose of levonorgestrel is to be administered in conjunction with other active ingredients including an estrogen.

Other progestins which can be used in part or total, in combination with estrogen, are norgestrel, norgestimate, desogestrel, gestodene, norethindrone, norethynodrel, hydrogesterone, ethynodiol diacetate, hydroxyprogesterone caproate, medroxyprogesterone acetate, norethindrone acetate, progesterone, megestrol acetate, gestogen and certain others which are biocompatible, absorbable transdermally, including biocompatible derivatives of progestins which are transdermally absorbed, desirably such derivatives which are bioconvertible after transdermal absorption to the original progestin. The progestin and estrogen hormones should have high compatibility with each other.

The effective dose of estrogen for inhibiting ovulation will depend upon the particular estrogen being co-administered. For instance, in some aspects, when the estrogen is ethinyl estradiol (EE), the dose will be at least 4 mcg/day, and in other aspects, from about 4 mcg/day to 28 mcg/day. In some embodiments, the dose will be from about 4 mcg/day to about 36 mcg/day. The patches will contain sufficient amounts of ethinyl estradiol to provide such daily doses for the intended patch wear time. Typically, such doses are from about 4 mcg/day. 5 mcg/day, 6 mcg/day, 7 mcg/day, 8 mcg/day, 9 mcg/day, 10 mcg/day, 11 mcg/day, 12 mcg/day, 13 mcg/day, 14 mcg/day, 15 mcg/day. 16 mcg/day, 17 mcg/day, 18 mcg/day, 19 mcg/day, 20 mcg/day, 21 mcg/day, 22 mcg/day. 23 mcg/day, 24 mcg/day, 25 mcg/day, 26 mcg/day, 27 mcg/day, 28 mcg/day, 29 mcg/day. 30 mcg/day, 31 mcg/day, 32 mcg/day, 33 mcg/day. 34 mcg/day, 35 mcg/day to about 36 mcg/day, in various embodiments, from about 8 mcg/day to about 36 mcg/day, and in other embodiments from about 12 mcg/day to about 32 mg/day of ethinyl estradiol. In some aspects, the ethinyl estradiol can be micronized. In some embodiments, the effective dose of ethinyl estradiol is to be administered alone. In some embodiments, the effective dose of ethinyl estradiol is to be administered in conjunction with other active ingredients including a progestin.

Other estrogens that may be combined with a progestin in the matrix include 17-β-estradiol and esters thereof such as estradiol valerate, estradiol cypionate, estradiol acetate, estradiol benzoate, and ethinyl estradiol. Ethinyl estradiaol (EE) is a preferred estrogen for use in combination with norelgestromin. EE/norelgestromin combinations may favorably affect metabolic parameters such as elevation of serum high density lipoprotein and reduction of the low density lipoprotein/high density lipoprotein ratio in serum.

When a transdermal patch is worn for contraception, the patch will typically be placed on the skin on the first day of the menstrual cycle and replaced as needed until 21 days of wearing have elapsed. For instance, in the case of a 7-day patch, three patches will be required to deliver the drug(s) for the 21-day period. If desired, a placebo patch may be worn thereafter until the fifth day of the succeeding menstrual cycle. This regimen is repeated for each menstrual cycle. A kit comprising one or more 7-day patches, the required prescribing information, and optionally additional materials such as a placebo patch, may be assembled in a single carton to aid in patient compliance for the duration of one or more menstrual cycles.

The transdermal system of this application can be in the form of a patch or a film. Patches or films of this application comprise a matrix and are monolithic-type laminated structures. They comprise a matrix of the drug(s) admixed with a pressure sensitive adhesive and a backing. The matrix serves as both the drug reservoir and the means by which the patch or film is affixed to the skin. Prior to use, the transdermal system will also include an impermeable release liner layer. The release liner contacts the matrix and is configured to be removed from the matrix. By using the transdermal system of this application, the pregnancy of a female patient is prevented even though she is at a decreased risk of VTEs relative to a woman using a transdermal system delivering higher amounts of EE.

Each transdermal system, in some embodiments, contains two active pharmaceutical agents, such as norelgestromin and ethinyl estradiol, which are dissolved in a pressure-sensitive adhesive matrix, and is designed to deliver one or more active agents transdermally.

The transdermal system of the current application allows delivery of about 4 to 30 mcg/day of an estrogen to the patient, while the delivery of other optional active agents of the transdermal delivery system, such as for example, progestin, are not interfered with. Thus, the patient can have consistent release of both estrogen and progestin, even though the dose of estrogen is lower.

The backing of the transdermal system is impermeable to the drug and other components of the matrix and defines the top face surface of the patch. It may be made of a single layer or film of polymer or be a laminate of one or more polymer layers and metal foil. Examples of polymers suitable for use in making backing films include without limitation polyvinylchloride, polyvinylidene chloride, polyolefins such as ethylene-vinyl acetate copolymers, polyethylene, and polypropylene, polyurethane, and polyesters such as polyethylene terephthalate. In many aspects, the backing is impermeable to both estrogen, for example, ethinyl estradiol and/or progestin, for example norelgestromin.

The pressure-sensitive adhesive of the drug reservoir matrix will normally be prepared from a solution of polyacrylate, a silicone, or polyisobutylene (PIB). Such adhesives are well known in the transdermal art. Sec, for instance, the Handbook of Pressure Sensitive Adhesive Technology. 2nd Edition (1989) Van Nostrand, Reinhold.

Pressure sensitive solution polyacrylate adhesives are made by copolymerizing one or more acrylate monomers (“acrylate” is intended to include both acrylates and methacrylates), one or more modifying monomers, and one or more functional group-containing monomers in an organic solvent. The acrylate monomers used to make these polymers include alkyl acrylates of 4-17 carbon atoms, with 2-ethylhexyl acrylate, butyl acrylate, and, in some embodiments, isooctyl acrylate. Modifying monomers are typically included to alter the Tg of the polymer. Such monomers as vinyl acetate, ethyl acrylate and methacrylate, and methyl methacrylate are useful for this purpose. The functional group-containing monomer provides sites for crosslinking. The functional groups of these monomers are, in many aspects, carboxyl, hydroxy or combinations thereof examples of monomers that provide such groups are acrylic acid, methacrylic acid and hydroxy-containing monomers such as hydroxyethyl acrylate. In various embodiments, the polyacrylate adhesives are crosslinked using a crosslinking agent to improve their physical properties. (e.g., creep and shear resistance). The crosslinking density should be low since high degrees of crosslinking may affect the adhesive properties of the copolymer adversely. Examples of crosslinking agents are disclosed in U.S. Pat. No. 5,393,529. Solution polyacrylate pressure sensitive adhesives are commercially available under tradenames such as GELVA™ and DURO-TAK™ from Henkel.

Polyisobutylene (PIB) adhesives are mixtures of at least one high molecular weight (HMW) PIB and at least one low molecular weight (LMW) PIB. Such mixtures are described in the art, e.g., PCT/US91/02516. Each high molecular weight polyisobutylene may have an average molecular weight of 500.000 to 1.5 million, or from 750,000 to 1.2 million. Each low molecular weight polyisobutylene may have an average molecular weight of 40,000 to 85,000.

Suitable polyisobutylene adhesives are commercially available. Alternatively, a suitable adhesive can be made by mixing a LMW PIB polymer with a HMW PIB polymer. In one embodiment, OPPANOL® N80 (HMW PIB) and OPPANOL® B12 (LMW PIB) may be used. In another embodiment, OPPANOL® N100 (HMW PIB) and OPPANOL® B10 (LMW PIB) may be used. For adhesives using a mixture of high and low molecular weight PIBs, the dry weight ratio of low molecular weight to high molecular weight PIB will normally range from 15:1, 14:1, 13:1, 12:1, 11:1, 10:1, 9:1, 8:1, 7:1, 6:1, 5:1, 4:1, 3:1, 2:1, 1:1. The molecular weights referred to herein are weight average molecular weight. Additionally, a mixture of adhesives may be used to achieve the desired adhesion and flow throughout the patch. The adhesives may be present in a dry weight ratio of from 15:1, 14:1, 13:1, 12:1, 11:1, 10:1, 9:1, 8:1, 7:1, 6:1, 5:1, 4:1, 3:1, 2:1, 1:1.

The silicone adhesives that may be used in forming the matrix are typically high molecular weight polydimethyl siloxanes or polydimethyldiphenyl siloxanes. Formulations of silicone adhesives that are useful in transdermal patches are described in U.S. Pat. Nos. 5,232,702, 4,906,169 and 4,951,622. In various aspects, the adhesive comprises, consists essentially of or consists of a solution of polyacrylate, a silicone or polyisobutylene.

The PIB adhesive may also comprise a tackifier such as polybutene oil, a plasticizer such as mineral oil, or a high Tg, low molecular weight aliphatic resins such as the ESCOREZ™ resins available from Exxon Chemical.

In various embodiments, the adhesive is in the matrix in an amount of about 58% w/w to about 58.05% w/w based on a total % w/w of the matrix. In other embodiments, the transdermal system is in a patch or film form and the adhesive is in the matrix in an amount of about 121.8 mg to about 121.9 mg per patch or film. In one aspect, the adhesive is in the matrix in an amount of about 58.07% w/w based on a total % w/w of the matrix. In another aspect, the adhesive is in the matrix in an amount of about 87.11 wt. g/m² based on a total wt. g/m² of the matrix.

In addition to the pressure sensitive adhesive, estrogen, and optional norelgestromin, the matrix will typically contain sufficient amounts of permeation enhancers to increase the permeability of the norelgestromin and estrogen through the skin and provide fluxes in the ranges described above. Examples of skin permeation enhancers that may be included in the matrix are described in U.S. Pat. Nos. 5,059,426; 4,973,468; 4,906,463; and 4,906,169, and include, but are not limited to lactate ester of C12 to C18 aliphatic alcohol, lauryl lactate, oleic acid, oleyl alcohol, or propylene glycol monolaurate (PGML). The amount of permeation enhancer included in the matrix will depend upon the particular enhancer(s) used. In most instances, the enhancer will constitute in the range of 1 to 20% by weight of the matrix.

Other permeation enhancers include, but are not limited to, polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethylidecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In one embodiment, the permeation enhancer is oleyl alcohol. In another embodiment, the penetration enhancer is a glycol, such as dipropylene glycol, propylene glycol, butylene glycol or polyethylene glycol. In other embodiments, the penetration enhancer comprises a mixture of at least two penetration enhancers.

In various embodiments, the matrix may contain other additives depending upon the particular adhesive used. For instance, materials, such as polyvinyl pyrrolidone (PVP), that inhibit drug crystallization, hygroscopic agents that improve the duration of wear, or additives that improve the physical (e.g., cold flow) or adhesive (e.g., tack, cohesive strength) properties of the matrix may be included.

A crystallization inhibitor or solubility enhancer may also be employed in the current application, for example polyvinylpyrrolidone polymers, polyethylene oxide, polyacrylic acid, polyvinyl alcohol, silicone dioxide, silica, celluloses and cellulose derivatives such as hydroxymethyl cellulose, hydroxypropyl cellulose, gelatins, gums, starches, dextrins and dextrans, sterols, bile acids and other absorptive agents that possess the capability to absorb and hold water or moisture.

Particularly preferred compounds are PVPs. The term “polyvinylpyrrolidone” or “PVP” refers to a polymer, ether a homopolymer or copolymer, containing vinylpyrrolidone (also referred to as N-vinylpyrrolidone, N-vinyl-2-pyrrolidone and N-vinyl-2-pyrrolidinone) as a monomeric unit. PVP polymers include soluble and insoluble homopolymeric PVPs, and copolymers such as vinylpyrrolidone/vinyl acetate and vinylpyrrolidone/dimethylamino-ethylmethacrylate. The cross-linked hompolymer is insoluble and is generally known in the pharmaceutical industry under the designations polyvinylpolypyrrolidone, crospovidone, and PVP.

PVPs are sold to the pharmaceutical industry under the trademarks KOLLIDON® by BASF (Parsippany, N.J.); PLASDONE™, POLYPLASDONE™ and COPOLYMER 958 by ISP Technologies (Wayne, N.J.) Other PVPs are KOLLIDON® CL-F, KOLLIDON® CL-SF, and KOLLIDON® CL-M.

Typically, the PVP is present in an amount from about 5% to about 50% by weight, preferably from about 10% to about 40% by weight based on the dry weight of the total adhesive matrix composition. However, the amount of PVP can be higher than 20% for example, up to 40%, depending on the particular drug used and on the desired properties of the matrix blend.

The release rate or delivery rate of the active from the transdermal system, onset of delivery (lag time) and delivery profile of the drug may be selectively modulated by one or more of (a) increasing or decreasing the thickness or coat weight of the acrylic-based adhesive coating per cm2 as applied to the backing of the system, (b) manipulating the moiety or functionality of the acrylic-based adhesive coating, and (c) manipulating the monomeric composition and/or ratios of the acrylic-based adhesive coating. Either the non-drug containing coating or the carrier composition must also be a pressure-sensitive adhesive when used as area of attachment to the skin or mucosa of the user. The drug carrier composition may be comprised of (a) one or more acrylic-based polymers having one or more functionality alone or in combination with (b) one or more silicone-based polymers having one or more silanol contents (capping) and/or resin to polymer ratios, and are present in proportions to provide a desired solubility for the drug. Further manipulation of drug delivery, onset and profiles can be achieved by varying the concentrations of the drug in the drug-loaded carrier.

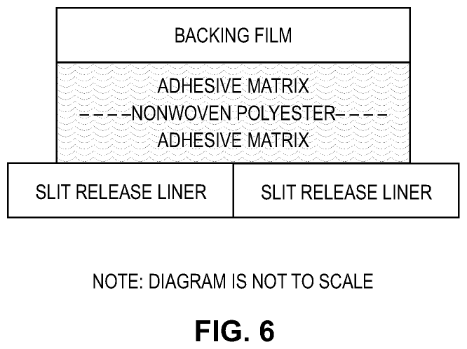

FIG. 6 is a schematic illustration of a representative transdermal system having norelgestromin in an amount of about 4.86 mg and ethinyl estradiol in an amount of about 0.264 mg. The outermost backing is a polyethylene/polyester film. The middle layer is the polyisobutene adhesive matrix containing the two active pharmaceutical ingredients, norelgestromin and ethinyl estradiol. It also contains several inactive ingredients, namely oleyl alcohol, dipropylene glycol, crospovidone, nonwoven polyester, and mineral oil. The third layer is a release liner that is slit near the middle to facilitate removal prior to use. This release liner is a transparent, fluoropolymer coated polyester film and both pieces are removed from the patch and discarded prior to use.

One embodiment of the transdermal delivery system comprises a 14 cm2 or less transdermal system, optionally with rounded corners, comprising or consisting of a backing film, an adhesive layer containing nonwoven fabric, and a clear oversized removable release liner, and further comprising 4.86 mg norelgestromin and 0.21 mg cthinyl estradiol. Each individual transdermal system is placed between two pieces of protective film and packaged in a sealed pouch which is imprinted with lot number and manufacturing date. In some embodiments, the transdermal delivery system comprises about 10 to about 28 mcg ethinyl estradiol and about 175 to about 225 meg norelgestromin. In some embodiments, the transdermal delivery system comprises about 12 to about 27 mcg ethinyl estradiol and about 190 to about 220 mcg norelgestromin. In some embodiments, the transdermal delivery system comprises about 19 to about 22 mcg ethinyl estradiol and about 199 to about 210 meg norelgestromin.

Each transdermal system contains, in some embodiments, two active pharmaceutical ingredients, norelgestromin (NGMN) and ethinyl estradiol (EE), that are dissolved into a pressure-sensitive adhesive matrix, and each is designed to deliver norelgestromin and ethinyl estradiol transdermally. The transdermal delivery system of this application contains, in some embodiments, three layers, wherein the matrix comprises norelgestromin 4.86 mg and ethinyl estradiol 0.21 mg. In some embodiments, the transdermal system can release estrogen in an amount about 17.5 mcg per day to about 28 meg per day. In some embodiments, the matrix of the transdermal system contains ethinyl estradiol in an amount of about 0.1% % w/w to about 0.19% w/w based on a total % w/w of the matrix.

In some embodiments, the transdermal system can release estrogen from the transdermal system in an amount from about 14 meg per day to about 28 per day. In some embodiments, the transdermal system can release estrogen from the transdermal system in an amount from about 14 meg per day, 14.5 mcg per day, 15 meg per day, 15.5 mcg per day, 16 meg per day, 16.5 meg per day, 17 mcg per day, 17.5 mcg per day. 18 meg per day, 18.5 mcg per day, 19 meg per day, 19.5 meg per day, 20 meg per day, 20.5 mcg per day, 21 meg per day, 21.5 mcg per day. 22 meg per day, 22.5 mcg per day, 23 meg per day. 23.5 meg per day, 24 meg per day, 24.5 mcg per day, 25 meg per day, 25.5 mcg per day, 26 mcg per day, 26.5 mcg per day, 27 meg per day, 27.5 mcg per day, 28 mcg per day, 28.5 mcg per day. 29 meg per day, 29.5 mcg per day, 30 meg per day, 30.5 meg per day, 31 meg per day to about 31.5 per day. In some embodiments, the estrogen (e.g., cthinyl estradiol) can be in the transdermal systems (e.g., transdermal film or patch) as the only active pharmaceutical ingredient.

In other aspects, the transdermal system is in a patch or film form and contains ethinyl estradiol in the matrix in an amount of about 0.21 mg to about 0.396 mg per patch or film. In the matrix, in some aspects, the ethinyl estradiol can be in an amount of about 0.130% w/w based on a total % w/w of the matrix. In other aspects, the amount of ethinyl estradiol in the matrix is in an amount of about 0.189 wt. g/m² based on a total wt. g/m² of the matrix.

In some embodiments, the transdermal system comprises ethinyl estradiol in the matrix in an amount from about 0.211 mg to about 0.320 mg. In some embodiments, the ethinyl estradiol is in the matrix in an amount from about 0.2112 mg to about 0.3168 mg. In some embodiments, the ethinyl estradiol is in the matrix in an amount from about 0.211 mg. 0.215 mg. 0.220 mg. 0.225 mg, 0.230 mg, 0.235 mg, 0.240 mg, 0.245 mg, 0.250 mg, 0.255 mg, 0.260 mg, 0.265 mg, 0.270 mg. 0.275 mg. 0.280 mg. 0.285 mg. 0.290 mg. 0.295 mg. 0.300 mg. 0.305 mg. 0.310 mg. 0.315 mg, to about 0.320 mg.

In some embodiments, the matrix of the transdermal system contains ethinyl estradiol in an amount of about 0.104% w/w to about 0.190% w/w based on a total % w/w of the matrix. In some embodiments, the matrix of the transdermal system contains ethinyl estradiol in an amount of about 0.105% w/w, 0.110% w/w, 0.115% w/w, 0.120% w/w, 0.125% w/w, 0.130% w/w. 0.135% w/w, 0.140% w/w, 0.145% w/w, 0.150% w/w, 0.155% w/w, 0.160% w/w, 0.165% w/w. 0.170% w/w, 0.175% w/w, 0.180% w/w, 0.185% w/w to about 0.190% w/w based on a total % w/w of the matrix.

In some embodiments, in addition to estrogen, the transdermal system comprises progestin (e.g., norelgestromin) in the matrix in an amount from about 3.6 mg to about 6.1 mg. In some embodiments, the norelgestromin is in the matrix in an amount from about 3.645 mg to about 6.075 mg. In some embodiments, the norelgestromin is in the matrix in an amount from about 3.6 mg, 3.65 mg. 3.7 mg, 3.75 mg. 3.8 mg. 3.5 mg. 3.9 mg, 3.95 mg, 4.0 mg. 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg. 5.8 mg, 5.89 mg, 5.9 mg. 6.0 mg to about 6.075 mg per patch or film.

In some embodiments, the matrix of the transdermal system contains progestin (e.g., norelgestromin) in an amount of about 1.7325% w/w to about 2.772% w/w based on a total % w/w of the matrix. In some embodiments, the matrix of the transdermal system contains norelgestromin in an amount of about 1.7% w/w, 1.7325% w/w, 1.8% w/w, 1.848% w/w, 1.9% w/w, 2.0% w/w, 2.1% w/w, 2.2% w/w, 2.31% w/w, 2.4% w/w, 2.5% w/w, 2.6% w/w, 2.7% w/w, 2.772% w/w, 2.8% w/w to about 2.8875% w/w based on a total % w/w of the matrix.

The transdermal system (e.g., patch or film) can be applied to the patient (e.g., mammal). The term “mammal” refers to organisms from the taxonomy class “mammalian” including, but not limited to, humans, other primates such as monkeys, chimpanzees, apes, orangutans and monkeys, rats, mice, rabbits, cats, dogs, pigs, cows, horses, etc. In some embodiments, the transdermal system (e.g., patch or film) can be applied to a human patient, such as a woman. In some embodiments, the patient is a human female.

The PK profile for the norelgestromin and ethinyl estradiol transdermal system is different from the PK profile for oral contraceptives in that it has a higher steady state concentrations and a lower peak concentration. Area under the time-concentration curve (AUC) and concentration at steady state CSS for EE are approximately 60% higher in women using norelgestromin and ethinyl estradiol transdermal system compared with women using an oral contraceptive containing 35 mcg of EE. In contrast, the peak concentration (Cmax) for EE is approximately 25% lower in women using the norelgestromin and ethinyl estradiol transdermal system. It is not known whether there are changes in the risk of serious adverse events based on the differences in PK profiles of EE in women using norelgestromin and ethinyl estradiol transdermal system compared with women using oral contraceptives containing 30 mcg to 35 mcg of EE. Increased estrogen exposure may increase the risk of adverse events, including VTE. (Xulane® Prescribing Information 2020).

The coat weight of the adhesive ranges from 100-200 g/m². In some embodiments, the coat weight may be 125-175 g/m², 140-160 g/m², or 145-155 g/m².

The transdermal system of this application has constant estrogen release at about 48 hours to about 168 hours after the transdermal system is applied to a skin of the patient. In some embodiments, as illustrated in the examples of this application, the transdermal system is configured to release over a period of seven days the ethinyl estradiol to produce an AUC∞ of about 6333.5 pg·hr/mL to about 9375.7 pg·hr/mL, a Cₘₐₓ of about 49.88 pg/mL to about 73.66 pg/mL, and a t_{1/2} about 17.65 hours to about 18.27 hours. In other embodiments, the transdermal system is configured to release over a period of seven days the ethinyl estradiol to produce an AUCₜₐᵤ of about 4695.6 pg·hr/mL to about 5522.2 pg·hr/mL and a Cₘₐₓ from 38.06 pg/mL to about 43.46 pg/mL.

In various embodiments of this application, the matrix of the transdermal system contains estrogen and a progestin, for example, norelgestromin. In some aspects, the norelgestromin is released from the transdermal system in an amount of about 150 meg per day. In some aspects, the norelgestromin is released from the transdermal system in an amount of about 200 mcg per day. In other aspects, the norelgestromin is in the matrix in an amount of about 2.31% w/w based on a total % w/w of the matrix.

In some aspects, the transdermal system is in a patch or film form and has estrogen and the norelgestromin is in the matrix in an amount of about 4.86 mg per patch or film. In other aspects, the norelgestromin is in the matrix in an amount of about 3.47 wt. g/m² based on a total wt. g/m² of the matrix.

In many aspects, as illustrated in the examples of this application, the transdermal system is configured to release over a period of seven days the norelgestromin to produce an AUC∞ of about 161928.6 pg·hr/mL to about 166150.0 pg·hr/mL, a Cₘₐₓ of about 1133.7 pg/mL to about 1117.6 pg/mL and a t_{1/2} about 28.73 hours to about 28.02 hours. This is when in combination with an estrogen.

In other embodiments, the transdermal system is configured to release over a period of seven days the norelgestromin to produce an AUCₜₐᵤ of about 132754.4 pg·hr/mL to about 155284.0 pg·hr/mL and a Cₘₐₓ of about 1033.5 pg/mL to about 1191.9 pg/mL. This is when in combination with an estrogen.

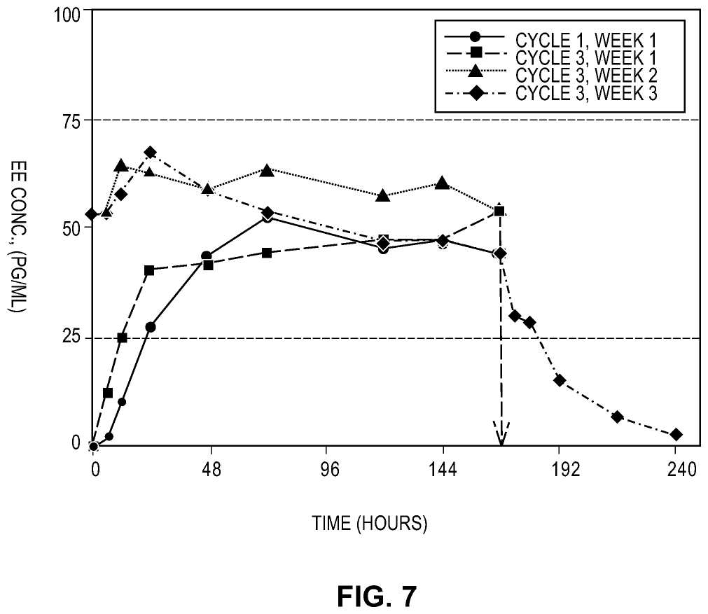

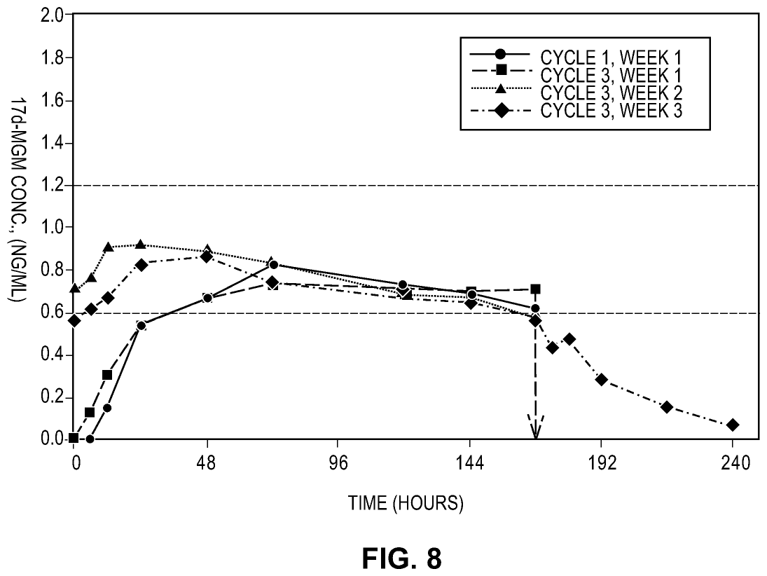

The release profiles for commercially available transdermal systems is shown in FIGS. 7 and 8. FIG. 7 is a graph illustrating mean EE serum concentrations in pg/mL in healthy female volunteers following application of a commercially available Ortho Evra® transdermal system applied to the buttocks for three consecutive cycles. The dotted horizontal lines indicate the reference range. The dotted vertical arrow indicates time of patch removal.

FIG. 8 is a graph illustrating mean NGMN serum concentrations in ng/ml in healthy female volunteers following application of a commercially available Ortho Evra® transdermal system applied to the buttocks for three consecutive cycles. The dotted horizontal lines indicate the reference range. The dotted vertical arrow indicates time of patch removal.

In many aspects, the transdermal system described in this application is applied to the skin of the patient in a regimen comprising application of one transdermal system once each week for three consecutive weeks. In other aspects, the transdermal system is applied to skin of the patient in a regimen comprising application of one transdermal system once each week for three consecutive weeks, followed by one week in which the transdermal system is not applied.

The transdermal system (e.g., film, patch, etc.) of the current application can be used to reduce, inhibit or prevent conception. The term “conception” is used to describe a deliberate prevention of conception or impregnation; or the deliberate use of artificial methods or other techniques to prevent pregnancy. In some embodiments, providing contraception is used to described deliberately providing an artificial means to prevent, or attempt to prevent pregnancy. Thus, in some embodiments, any device (e.g., transdermal system of the present application) or act whose purpose is to prevent a woman from becoming pregnant can be considered as a contraceptive.

The transdermal system (e.g., film, patch, etc.) of the current application can be used to block or inhibit the process that leads to ovulation. Both estrogens and progestins can function to inhibit ovulation. Estrogens suppress Follicle Stimulating Hormone, preventing development of a dominant follicle that ultimately leads to ovulation. Progestins suppress Luteinizing Hormone, blocking ovulation. In addition, progestins thicken the cervical mucus, reduce ovum movement, and thin the endometrium, thereby reducing the likelihood of implantation. Therefore, ovulation inhibitors can be used to treat diseases or conditions including, but not limited to, polycystic ovarian syndrome, endometriosis, endometrial hyperplasia, menorrhagia, endometriosis, menopausal hormone therapy, dysmenorrhea, dysfunctional uterine bleeding, acne or a combination thereof.

The transdermal system (e.g., film, patch, etc.) of the current application can lower the risk of venous thromboembolism events as compared to a patient receiving more than 28 mcg per day of estrogen. The risk can be lowered, in some embodiments, by 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to a patient receiving more than 28 meg per day of estrogen.

In some embodiments, there is a method for blocking ovulation in a patient in need thereof, the method comprising providing a transdermal system for releasing an estrogen to a patient in need thereof, the transdermal system, such as a transdermal system described herein, comprising a backing, and a matrix contacting the backing, the matrix configured to release about 4 meg per day to about 28 meg per day of the estrogen to the patient and applying the transdermal system to skin of the patient. In some embodiments, there is a method for providing estrogen therapy or estrogen therapy with progestin therapy to a patient in need thereof, the method comprising providing a transdermal system, such as a transdermal system described herein, for releasing an estrogen to a patient in need thereof, the transdermal system comprising a backing, and a matrix contacting the backing, the matrix configured to release about 4 meg per day to about 28 meg per day of the estrogen to the patient and applying the transdermal system to skin of the patient. In certain embodiments of these methods and the transdermal systems of the invention used therefore, the transdermal system provided to the patient further comprises a therapeutically effective amount of a progestin. The conditions that can benefit from blocking ovulation include but are not limited to, polycystic ovarian syndrome, endometriosis, endometrial hyperplasia, menorrhagia, endometriosis, menopausal hormone therapy, dysmenorrhea, dysfunctional uterine bleeding, acne or a combination thereof. These are conditions that are responsive to estrogen treatment or responsive to treatment with both estrogen and progestin.

“Treating” or “treatment” of a disease or condition refers to executing a protocol that may include administering the transdermal system of the current application to a patient (human, other normal or otherwise or other mammal), in an effort to inhibit ovulation, prevent pregnancy, or provide estrogen, or estrogen and progestin. A “therapeutically effective amount” or “effective amount” is such that when administered, the drug results in alteration of the biological activity, such as, for example, inhibition of ovulation, prevention of pregnancy, or provide estrogen, or estrogen and progestin.

The transdermal system (e.g., film, patch, etc.) of the current application can be made, in some embodiments of the application, by first preparing separate adhesive blends for each layer of the dosage unit, then dissolving or suspending the estrogen, or estrogen and progestin in at least one of the blends, each of which has been made by mixing a suitable solvent with the pressure sensitive adhesive of choice. The anchor layer is coated first on a release liner, dried and then laminated to the desired backing film, according to predetermined parameters, such as temperature and dwell time (line speed), which yield minimal residual solvent levels. The skin contact layer then is coated on a separate release liner and dried. The release liner is removed from the anchor layer and the adhesive side of the skin contact layer is laminated onto the adhesive side of the anchor layer so that the anchor layer is between the backing and the skin contact layer. If the estrogen, or estrogen and progestin initially is suspended or dissolved in only one of the two adhesive layers, it will, over time, equilibrate into the other adhesive layer until a common equilibrium is achieved. In some embodiments, the estrogen, or estrogen and progestin can be initially suspended or dispersed in only one of the two adhesive layers if, for example, the other adhesive layer is prepared with a solvent which would be deleterious to the drug but which evaporates during processing (coating and drying).

If more than two layers are to be provided, the third (middle) layer is coated as a liquid onto a release liner, dried, laminated to either the adhesive side of the dried skin contact layer or the adhesive side of the dried anchor layer once the release liner has been removed from the latter, then the two parts of the dosage unit are laminated to one another as above.

Suitable solvents for use in preparing the adhesive blends include acetone, heptane, ethyl acetate, isopropanol, ethanol, hexane, toluene, xylene, 2,4-pentanedione, methanol and water.

Alternative methods for producing or achieving a transdermal delivery dosage unit in accordance with this disclosure may be apparent to persons skilled in the art, and such alternative methods also fall within the scope of the present application. For example, an adhesive blend can be coated onto the backing film rather than the release liner. Alternatively, an adhesive coating can be created without using a solvent, such by heating the adhesive to its melting temperature (hot-melt adhesive). With this technique, no drying of the adhesive is required, only cooling.

There are many coating techniques for applying a continuous liquid coating onto a substrate, including using a gravure roll, reverse roll, falling film, inkjet, etc. All of these are well-known to persons of ordinary skill in the art and can be used to create pressure-sensitive adhesive layers from a solvated blend. Alternatively, a thin adhesive coating can be achieved by extrusion, in which the adhesive blend is forced through a die under pressure onto the substrate either as a continuous coating or as a printed (intermittent) pattern.

The thickness of the anchor and skin contact layers of the compositions of this application can vary, depending upon such factors as the amount of drug to be delivered from the composition and the desired wear period. Generally, however, the skin contact layer has a thickness of between about 5 and 150 gsm, preferably between about 25 and 50 gsm. The anchor layer generally has a thickness of between about 5 and 150 gsm, preferably between about 25 and 100 gsm.

In some embodiments, the transdermal system comprises oleyl alcohol, dipropylene glycol, crospovidone, and/or mineral oil.

### EXAMPLES

The following examples further illustrate the application. These examples are not intended to limit the application in any manner. Unless indicated otherwise, stated percentages are by weight.

Some embodiments of the low dose transdermal patches or films described in this application are given in Examples 1-2.

### Example 1

This example illustrates two transdermal systems, having the pharmaceutical formulations as shown below.

| | Treatment A | | Treatment B | |
|---|---|---|---|---|
| | 0.264 mg EE/4.86 mg | | 0.396 mg EE/4.86 mg | |
| | norelgestromin | | norelgestromin | |
| Ingredients | % w/w | mg/patch | % w/w | mg/patch |

Active Ingredients

| | | | | |
|---|---|---|---|---|
| Norelgestromin, USP | 2.31 | 4.86 | 2.31 | 4.86 |
| Ethinyl estradiol, USP | 0.125 | 0.264 | 0.19 | 0.40 |

Inactive Ingredients

| | | | | |
|---|---|---|---|---|
| Polyisobutylene | 58.05 | 121.9 | 58.00 | 121.8 |
| adhesives | | | | |
| Oleyl alcohol | 3.55 | 7.45 | 3.55 | 7.45 |
| Dipropylene glycol | 0.75 | 1.58 | 0.75 | 1.58 |
| Light Mineral Oil | 12.57 | 26.40 | 12.57 | 26.40 |
| Crospovidone | 22.62 | 47.50 | 22.62 | 47.50 |
| Total Theoretical | 100.00 | 210.00 | 100.00 | 210.00 |
| Matrix | | | | |

Inactive Ingredients
Polyethylene/polyester backing
Nonwoven polyester (optional)
Release liner

The transdermal systems of the application may be fabricated using conventional procedures in the transdermal delivery system art, such as those described in U.S. Pat. No. 10,632,082. The procedure will generally involve formulating the matrix (i.e., mixing the adhesive, drug(s), permeation enhancer, and additives, if any), casting the matrix onto the backing or release liner layer, removing solvent from the matrix and applying the backing/release liner layer as the case may be. As is apparent to those of ordinary skill in the art, the matrix composition having an effective amount of the drug dispersed therein can be incorporated into various transdermal constructions and therefore, applicants are not limited to the embodiments exemplified below.

The method of manufacture of the low dose transdermal films of Example 1 includes: (1) dispensing and mixing; (2) first-pass coating: (3) second pass coating; (4) slitting; and (5) die cutting and packaging.

The dispensing and mixing process involves making two identical blends of the active and inactive ingredients. One blend is coated to manufacture the first pass laminate, and the second blend is coated to manufacture the second pass laminate.

The first pass laminate and the second pass laminate are coated in a two-pass configuration where the first pass is laminated to the second pass, resulting in a bilayer laminate. Optionally, a scrim, optionally made from non-woven polyester, may be used in between the first pass coating and second pass coating.

Slitting of the bilayer laminate is performed by unwinding the bilayer laminate through a set of knives and rewinding to create several narrow, slit rolls of bilayer laminate. Optionally, knife spacing is adjusted to provide the desired final dimensions for further processing. Slitting is a common process, and standard slitting conditions known to a person of skill in the art were successfully used in the manufacture of this prototypical patch.

The die cutting operation determines the patch size and thus the dosage of the finished drug product. The parameter for the die-cutting step is patch dimension (length×width). Material thickness and physical properties of the backing are known to affect the performance of the kiss-cut die during packaging.

The backing used for the patches of Example 1 was pre-printed. Thus, the printing unit operation was not necessary for the production of Example 1. However, the use of such an operation is well known in the art.

A pharmacokinetic study was performed by comparing the pharmacokinetic characteristics of the Treatment A ethinyl estradiol transdermal system from Example 1 against TRI-LO-MARZIA™, an immediate release tablet containing 25 mcg ethinyl estradiol and 180 mcg norgestimate. TRI-LO-MARZIA™ tablets are commercially available in the U. S. and indicated for the prevention of pregnancy. The tablets were orally administered once/week for two cycles; the transdermal systems of Example 1 were transdermally administered once/week for three cycles at two different application sites, the abdomen and the buttocks. Each delivery system (e.g., film or patch) was worn for seven days, followed on the eighth day by a new film or patch.

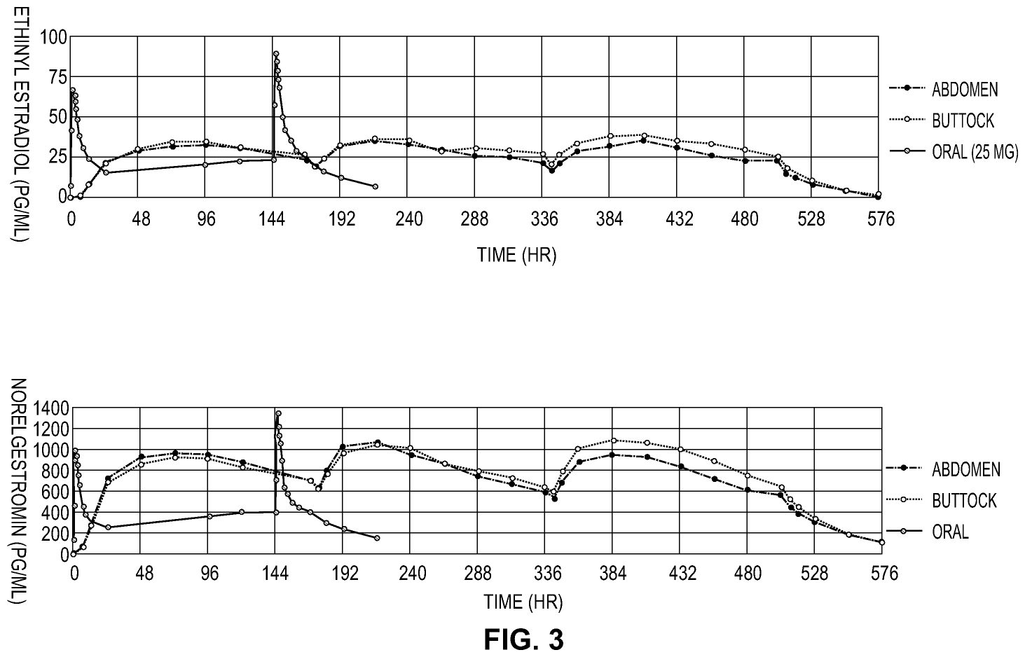

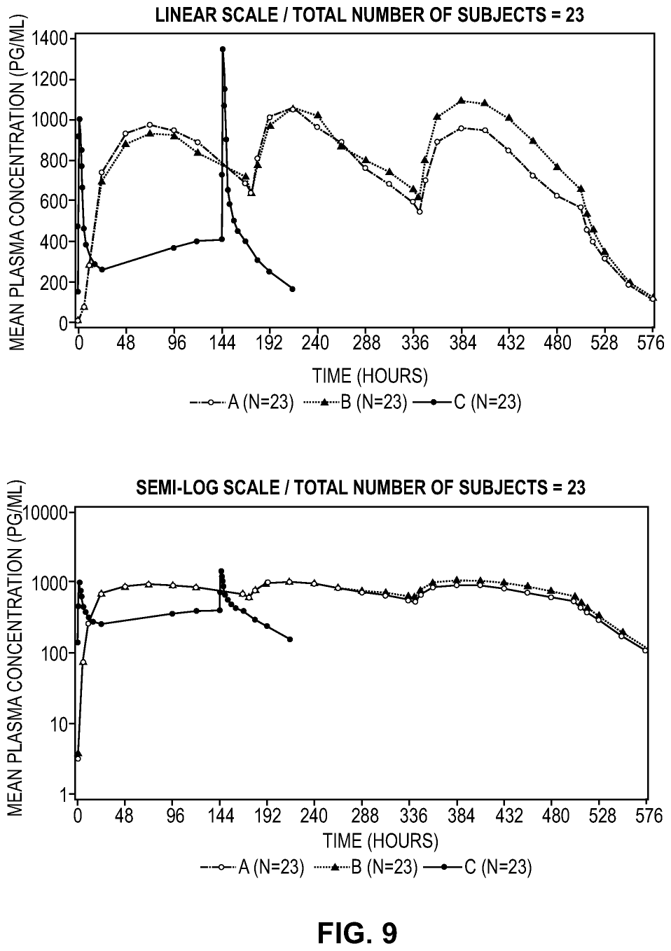

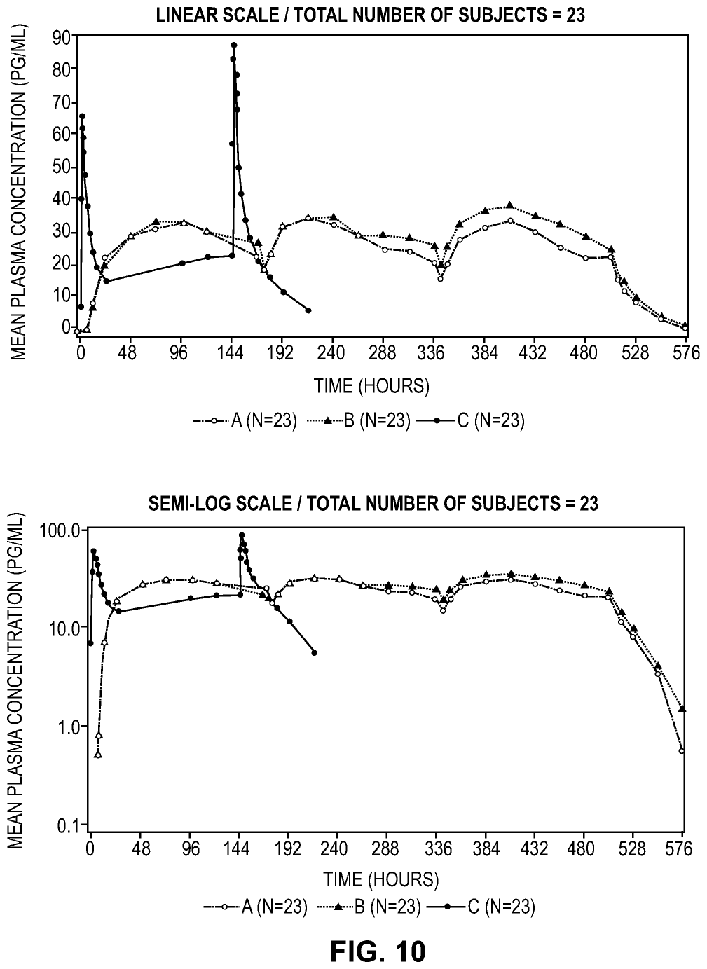

The pharmacokinetic data of the low dose transdermal systems are detailed in Table 1. A graphical representation of this data is presented as FIG. 3. FIG. 9 illustrates linear and semi log scale graphs of mean norelgestromin plasma concentrations produced by treatment groups A, B, and C of Table 1. FIG. 10 illustrates linear and semi log scale graphs of mean ethinyl estradiol plasma concentrations produced by treatment groups A, B, and C of Table 1.

The results illustrate a significant difference in pharmacokinetic profiles between the transdermal film and the immediate release tablet due to these different mechanisms of administration.

TABLE 1
Pharmacokinetic Data for Low Dose Transdermal System of Example 1
Mean Ethinyl Estradiol Pharmacokinetic Parameters
Arithmetic Mean (% CV) Ethinyl Estradiol Pharmacokinetic Parameters

| | Trt A = Mylan | Trt B = Mylan | Trt C = Tri-Lo-Marzia ™ |
|---|---|---|---|
| | Lot. No. 4000752 | Lot. No. 4000752 | Lot No. L801129 |
| | Abdomen | Buttocks | Oral |
| | Patch 3 (Day 15 to 22) | Patch 3 (Day 15 to 22) | Day 7 |
| Parameter | (n = 23) | (n = 23) | (n = 23) |
| AUCtau (pg*hr/mL) | 4695.6 (49.3%) | 5522.2 (35.4%) | 991.0 (51.2%) |
| CPEAK (pg/mL) | 38.06 (43.9%) | 43.46 (27.0%) | 94.02 (35.2%) |
| CMIN (pg/mL) | 13.88 (80.4%) | 16.65 (71.9%) | 23.17 (88.2%) |
| TPEAK (hr) | 80.79 (43.4%) | 75.89 (47.5%) | 1.449 (36.1%) |

Geometric LSMeans Ratios (90% Confidence Intervals) of the natural log-transformed PK parameters for
each test/reference comparison.

| | Treatment A/ | Treatment A/ | Treatment B/ |
|---|---|---|---|
| Parameter | Treatment B* | Treatment C* | Treatment C* |
| LNAUCtau (pg*hr/mL)^{b} | 0.86 | 0.69 | 0.79 |
| | (69.12%-106.00%) | (56.30%-83.50%) | (65.16%-96.02%) |
| LNCPEAK (pg*hr/mL) | 0.90 | 0.42 | 0.47 |
| | (79.06%-101.87%) | (37.43%-48.00%) | (41.46%-52.94%) |
| LNCMIN (pg/mL) | 0.72 | 0.88 | 1.09 |
| | (52.57%-98.23%) | (70.27%-109.69%) | (87.59%-136.73%) |

*Ratio (Test/Reference) = e^{[LSMEAN of (LNTEST-LNReference)]}
^{a}n = 22, Subject 11 had no EE plasma levels for Patch 3, Treatment A
^{b}The LNAUCtau comparison for A/C and B/C was made by multiplying the Day 7 0-24 hr AUCtau times 7 for
Treatment C, to compare with AUCtau 0-168 hr for Treatment A and B.

Mean Norelgestromin Pharmacokinetic Parameters
Arithmetic Mean (% CV) Norelgestromin Pharmacokinetic Parameters

| | Trt A = Mylan | Trt B = Mylan | Trt C = Tri-Lo-Marzia ™ |
|---|---|---|---|
| | Lot. No. 4000752 | Lot. No. 4000752 | Lot No. L801129 |
| | Abdomen | Buttocks | Oral |
| | Patch 3 (Day 15 to 22) | Patch 3 (Day 15 to 22) | Day 7 |
| Parameter | (n = 23) | (n = 23) | (n = 23) |
| AUCtau (pg*hr/mL) | 132754.4 (38.4%) | 155284.0 (31.7%) | 14428.0 (47.0%) |
| CPEAK (pg/mL) | 1033.5 (34.0%) | 1191.9 (24.2%) | 1493.3 (29.7%) |
| CMIN (pg/mL) | 464.9 (67.8%) | 509.0 (55.5%) | 392.9 (68.5%) |
| TPEAK (hr) | 63.39 (29.7%) | 62.83 (53.7%) | 1.428 (48.8%) |

Geometric LSMeans Ratios (90% Confidence Intervals) of the natural log-transformed PK parameters for each
test/reference comparison

| | Treatment A/ | Treatment A/ | Treatment B/ |
|---|---|---|---|
| Parameter | Treatment B* | Treatment C* | Treatment C* |
| LNAUCtau (pg*hr/mL)^{b} | 0.89 | 1.35 | 1.52 |
| | (76.61%-103.25%) | (113.31%-160.12%) | (127.87%-179.67%) |
| LNCPEAK (pg*hr/mL) | 0.90 | 0.72 | 0.80 |
| | (81.91%-99.34%) | (63.77%-81.40%) | (71.29%-90.63%) |
| LNCMIN (pg/mL) | 0.96 | 1.22 | 1.28 |
| | (62.85%-147.44%) | (85.56%-175.35%) | (90.28%-182.59%) |

*Ratio (Test/Reference) = e^{[LSMEAN of (LNTEST-LNReference)]}
^{a}n = 22, Subject 11 had no NGMN plasma levels for Patch 3, Treatment A
^{b}The LNAUCtau comparison for A/C and B/C was made by multiplying the Day 7 0-24 hr AUCtau times 7 for
Treatment C, to compare with AUCtau 0-168 hr for Treatment A and B.
Treatment A: Norelgestromin 4.86 mg/Ethinyl Estradiol 0.264 mg Transdermal System, worn on abdomen,
Lot #4000752, Mylan
Treatment B: Norelgestromin 4.86 mg/Ethinyl Estradiol 0.4 mg Transdermal System, worn on buttock,
Lot #4000752, Mylan
Treatment C: Tri-Lo-Marzia, Lot #L801129, Dose: 1 × 0.180 mg norgestimate/0.025 mg ethinyl estradiol
tablet/day for 7 days, Lupin

### Example 2

This example provides additional pharmacokinetic information for the transdermal systems described in this application. The PK data was obtained from 24 healthy women. In particular, a clinical study was performed on 24 healthy subjects who were included in the Treatment A PK population, 22 subjects were included in the Treatment B PK population, and 21 subjects were included in the Treatment C PK population for ethinyl estradiol. The subjects applied to skin a single patch application of Treatment A (Norelgestromin 4.86 mg/Ethinyl Estradiol 0.264 mg Transdermal System), Treatment B (Norelgestromin 4.86 mg/Ethinyl Estradiol 0.40 mg Transdermal System), and Treatment C (Mylan's Xulane® Transdermal System containing 4.86 mg NGMN and 0.53 mg EE), which was worn on the right or left side of the upper back for 168-hours in each study period. The pharmacokinetic results are summarized below.

### Norelgestromin:

Arithmetic Mean (% CV) Norelgestromin Pharmacokinetic Parameters in Healthy
Adult Female Subjects Following a Single Transdermal Application of Norelgestromin
and Ethinyl Estradiol Transdermal System Worn for 7 Days

| | Trt A = Mylan | Trt B = Mylan | Trt C = Xulane ® |
|---|---|---|---|
| | Lot No. 4000752 | Lot No. 4000710 | Lot No. 3093154 |
| Parameter | (n = 24) | (n = 24) | (n = 20) |
| AUCINF (pg*hr/mL) | 161928.6 (31.0%)¹ | 166150.0 (28.2%)¹ | 164153.1 (25.3%) |
| AUCL (pg*hr/mL) | 166897.0 (39.9%) | 166053.6 (30.1%) | 160177.4 (25.8%) |
| CPEAK (pg/mL) | 1133.7 (35.0%) | 1117.6 (30.5%) | 1113.1 (27.5%) |
| KEL (1/hr) | 0.0250 (20.1%)¹ | 0.0258 (19.4%)¹ | 0.0264 (22.7%) |
| HALFLIFE (hours) | 28.73 (19.4%)¹ | 28.02 (22.9%)¹ | 27.66 (24.0%) |
| TPEAK (hours) | 71.06 (32.4%) | 77.04 (26.1%) | 74.41 (44.4%) |

¹n = 23

### Ethinyl Estradiol:

Arithmetic Mean (% CV) Ethinyl Estradiol Pharmacokinetic Parameters in Healthy
Adult Female Subjects Following a Single Transdermal Application of Norelgestromin
and Ethinyl Estradiol Transdermal System Worn for 7 Days

| | Trt A = Mylan | Trt B = Mylan | Trt C = Xulane ® |
|---|---|---|---|
| | Lot No. 4000752 | Lot No. 4000710 | Lot No. 3093154 |
| Parameter | (n = 24) | (n = 22) | (n = 21) |
| AUCINF (pg*hr/mL) | 6333.5 (37.6%)¹ | 9375.7 (47.9%)² | 12466.8 (28.4%) |
| AUCL (pg*hr/mL) | 6602.7 (44.3%) | 9638.5 (48.6%) | 12320.2 (28.2%) |
| CPEAK (pg/mL) | 49.88 (35.4%) | 73.66 (47.5%) | 98.14 (30.1%) |
| KEL (1/hr) | 0.0411 (23.3%)¹ | 0.0424 (30.3%)² | 0.0420 (22.7%) |
| HALFLIFE (hours) | 17.65 (21.8%)¹ | 18.27 (41.4%)² | 17.37 (23.9%) |
| TPEAK (hours) | 100.0 (47.3%) | 89.49 (39.9%) | 104.1 (42.7%) |

¹n = 23,
²n = 21
Treatment A: Norelgestromin 4.86 mg/Ethinyl Estradiol 0.264 mg Transdermal System, Lot #4000752, Mylan
Treatment B: Norelgestromin 4.86 mg/Ethinyl Estradiol 0.40 mg Transdermal System, Lot #4000710, Mylan
Treatment C: Xulane ®, Norelgestromin 4.86 mg/Ethinyl Estradiol 0.53 mg Transdermal System, Lot #3093154, Mylan

Residual patch analysis performed on Treatment A unexpectedly revealed that Treatment A delivered about 19 mcg of EE/day and about 204 mcg NGM/day.

Adhesion results were also collected and analyzed according to FDA's “Assessing Adhesion With Transdermal and Topical Delivery Systems for ANDAs”, Draft Guidance for Industry, October 2018. The Guidance provides a method for scoring, on a scale of 0-4, the quality of adhesion over the proposed wear period of a patch. The scores can then be tabulated and converted into a single cumulative mean representing an adhesion score for the patch. It was found that the adhesion scores for Treatments A and B were very similar to the adhesion score for Xulane®.

Arithmetic Mean (% CV) of Adhesion Scores Observed in Healthy
Adult Female Subjects Following a Single Dose of Norelgestromin
and Ethinyl Estradiol Transdermal System Worn for 7 Days

| | Trt A = Mylan | Trt B = Mylan | Trt C = Xulane ® |
|---|---|---|---|
| | Lot No. 4000752 | Lot No. 4000710 | Lot No. 3093154 |
| | (n = 24) | (n = 24) | (n = 21) |
| Cumulative | 1.20 (41.0%) | 1.11 (44.0%) | 1.29 (38.7%) |
| Mean | | | |

Treatment A: Norelgestromin 4.86 mg/Ethinyl Estradiol 0.264 mg Transdermal System, Lot #4000752, Mylan
Treatment B: Norelgestromin 4.86 mg/Ethinyl Estradiol 0.40 mg Transdermal System, Lot #4000710, Mylan
Treatment C: Xulane ®, Norelgestromin 4.86 mg/Ethinyl Estradiol 0.53 mg Transdermal System, Lot #3093154, Mylan

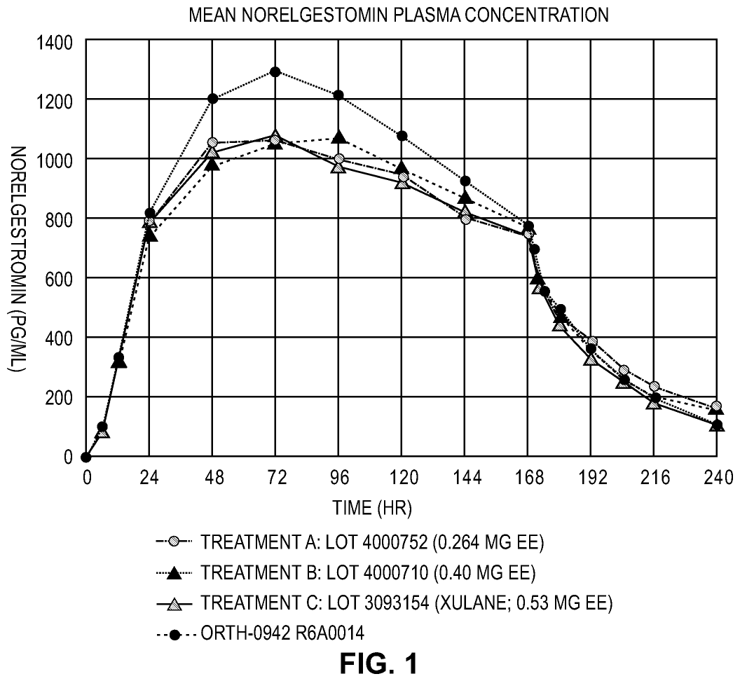

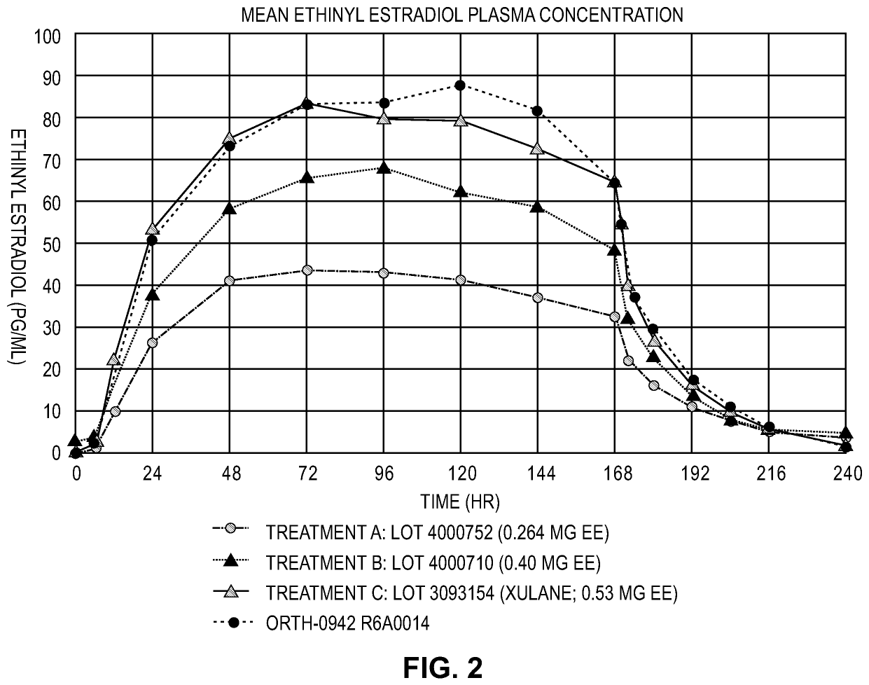

The information in this example for Treatments A, B and C is reflected in FIGS. 1 and 2. In FIG. 1, norelgestromin plasma concentrations produced by transdermal systems from Treatment A (a transdermal system comprising 4.86 mg norelgestromin and 0.264 mg ethinyl estradiol Treatment B (a transdermal system comprising 4.86 mg norelgestromin and 0.40 mg ethinyl estradiol, and Treatment C (XULANE® transdermal system containing 4.86 mg NGMN and 0.53 mg EE) are shown.

In FIG. 2, ethinyl estradiol plasma concentrations produced by transdermal systems from Treatment A, B, and C are shown.

The contemplated EE pharmacokinetic (PK) parameter ranges for Treatments A and B in Examples 1-2 are listed in Table 2.

TABLE 2

| PK Parameter | Minimum | Maximum |
|---|---|---|
| Cmax (pg/mL) | 14.61 | 87.81 |
| AUCtau (pg*hr/mL) | 716 | 9392 |
| Cmin (pg/mL) | 0.00 | 78.3 |

The PK parameters are not dependent on the particular transdermal application site (e.g., right or left side of the upper back, etc.). Cmin starts at the time the transdermal system is applied to skin and the PK parameter calculations include when the transdermal system is removed from the skin at 168 hours.

Although the invention has been described with reference to embodiments, persons skilled in the art will recognize that changes may be made in form and detail without departing from the spirit and scope of the disclosure.

1.-107. (canceled)

108. A transdermal system for delivering a contraceptive to a patient in need thereof, the transdermal system comprising a backing and a matrix contacting the backing, wherein the matrix delivers about 5 mcg per day to about 28 mcg per day of an estrogen to the patient.

109. The transdermal system of claim 108, wherein the estrogen is released from the transdermal system in an amount of about 17.5 mcg per day to about 26.25 mcg per day.

110. The transdermal system of claim 108, wherein the estrogen comprises ethinyl estradiol.

111. The transdermal system of claim 110, wherein the transdermal system is in a patch or film form and the ethinyl estradiol is in the matrix in an amount of about 0.21 mg to about 0.48 mg per patch or film.

112. The transdermal system of claim 110, wherein the transdermal system has constant estrogen release at about 48 hours to about 168 hours after the transdermal system is applied to a skin of the patient.

113. The transdermal system of claim 112, wherein the transdermal system is configured to release over a period of seven days the ethinyl estradiol to produce an AUC∞ about 6333.5 pg·hr/mL to about 9375.7 pg·hr/mL, a Cₘₐₓ about 49.88 pg/mL to about 73.66 pg/mL, and a t_{1/2} about 17.65 hours to about 18.27 hours.

114. The transdermal system of claim 112, wherein the transdermal system is configured to release over a period of seven days the ethinyl estradiol to produce an AUCₜₐᵤ about 4695.6 pg·hr/mL to about 5522.2 pg·hr/mL and a Cₘₐₓ from 38.06 pg/mL to about 43.46 pg/mL.

115. The transdermal system of claim 112, wherein the transdermal system is configured to release estrogen in an amount of (i) 5 to 28 mcg/day; (ii) about 10 to about 27 mcg/day; or (iii) about 15 to about 20 mcg/day.

116. The transdermal system of claim 110, wherein the matrix further comprises a progestin.

117. The transdermal system of claim 116, wherein the progestin comprises norelgestromin.

118. The transdermal system of claim 117, wherein the norelgestromin is released from the transdermal system in an amount from about 190 to about 210 mcg per day.

119. The transdermal system of claim 118, wherein the transdermal system is configured to release over a period of seven days the norelgestromin to produce an AUC∞ about 161928.6 pg·hr/mL to about 166150.0 pg·hr/mL, a Cₘₐₓ about 1133.7 pg/mL to about 1117.6 pg/mL and a t_{1/2} about 28.73 hours to about 28.02 hours.

120. The transdermal system of claim 119, wherein the transdermal system is configured to release over a period of seven days the norelgestromin to produce an AUCₜₐᵤ about 132754.4 pg·hr/mL to about 155284.0 pg·hr/mL and a Cₘₐₓ about 1033.5 pg/mL to about 1191.9 pg/mL.

121. The transdermal system of according to claim 108, wherein the system further comprises oleyl alcohol, dipropylene glycol, crospovidone, or mineral oil.

122. A method of making a transdermal system, the method comprising mixing about 0.21 mg to about 0.48 mg of estrogen with an adhesive and applying it to the transdermal system, wherein the transdermal system is configured to release about 4 mcg per day to about 26 mcg per day of an estrogen to a patient.

123. A method of providing an estrogen to a patient in need thereof, the method comprising applying to skin of the patient a transdermal system comprising a backing and a matrix contacting the backing, wherein the matrix delivers about 4 mcg per day to about 28 mcg per day of an estrogen to the patient, wherein the estrogen is the only active pharmaceutical ingredient in the transdermal system.

124. A transdermal system for releasing a contraceptive to a patient in need thereof, the transdermal system comprising a backing, and a matrix contacting the backing, the matrix configured to release about 4 mcg per day to about 28 mcg per day of an estrogen to the patient, wherein pregnancy of a human female patient is prevented and the human female patient has a body mass index (BMI) of less than 30 kg/m².

125. A transdermal system for releasing a contraceptive to a patient in need thereof, the transdermal system comprising a backing, and a matrix contacting the backing, the matrix configured to release about 4 mcg per day to about 28 mcg per day of an estrogen to the patient, wherein pregnancy of a human female patient is prevented and the human female patient has a body mass index (BMI) of greater than or equal to 30 kg/m².

126. A transdermal system for releasing a contraceptive to a patient in need thereof, the transdermal system comprising a backing, and a matrix contacting the backing, the matrix configured to release about 4 mcg per day to about 28 mcg per day of an estrogen to the patient, wherein the patient has a lower risk of venous thromboembolism events as compared to a patient receiving more than 28 mcg per day of estrogen.

127. A method of treating a condition responsive to an estrogen, the method comprising applying to skin of a patient a transdermal system comprising a backing, and a matrix contacting the backing, the matrix configured to release about 4 meg per day to about 28 meg per day of an estrogen to the patient.
